(19) United States
(12) Patent Application Publication
(10) Pub. No.: US 2026/0096864 A1
(43) Pub. Date: Apr. 9, 2026

(54) SURGICAL ROBOTIC SYSTEM AND METHOD FOR AUTOMATIC GRASPING FORCE ADJUSTMENT DURING SUTURING

(71) Applicant: Covidien LP, Mansfield, MA (US)

(72) Inventors: Gregory W. Fischvogt, Reno, NV (US); Jonathan D. Thomas, New Haven, CT (US)

(21) Appl. No.: 19/114,095

(22) PCT Filed: Sep. 25, 2023
(86) PCT No.: PCT/IB2023/059447

§ 371 Date: Mar. 21, 2025

Related U.S. Application Data
(60) Provisional application No. 63/410,718, filed on Sep. 28, 2022.

(51) Int. Cl. A61B 34/37; A61B 17/00; A61B 17/062; A61B 34/30; A61B 90/00

(52) U.S. Cl. CPC A61B 34/37; A61B 17/0625; A61B 90/06; A61B 2017/0003; A61B 2034/305; A61B 2090/066; A61B 2090/067

(57) ABSTRACT

A surgical robotic system includes a robotic arm having an instrument with a pair of opposing jaws configured for grasping, and an instrument drive unit configured to couple to and to actuate the instrument. The system also includes a surgeon console having a handle controller configured to control the robotic arm and the instrument. The system further includes a controller configured to: receive an electrical property of the opposing jaws; receive an angle of the opposing jaws; determine whether the opposing jaws are grasping a metallic object; and adjust a gripping force of the opposing jaws based on the determination.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Proliferation of robotically assisted wristed instruments resulted in the usage of suture-based repair in minimally invasive surgery. Sutures are commonly used to close abdominal wall defects in hernia procedures, attach hernia meshes to adjacent tissue without the use of tacks, perform hand-sewn anastomosis, close enterotomies, etc.

Current robotic systems do not have the ability to fully replicate the experience of suturing in an open surgical environment. Surgeons lack sufficient haptic feedback which is an essential element in operating on delicate structures. Surgeons rely only on visual cues to determine sensory inputs such as tissue compression and suture tension. These visual cues are often trailing indicators of damage to the tissue that can lead to ischemia, necrosis, and in some cases, inflammation, and adhesions.

Robotic system manufacturers are aware of this teleoperative issue and have begun to integrate sound or vibratory alarm features into their products that are triggered by machine measurements. Although a step in the right direction, this increases the cognitive load required by surgeons. They need to first sense the feedback (e.g., haptic vibration), then interpret, and finally react in response. In the time for the stimulus to register and the surgeon to react, the damage to suture or tissue may have already been done.

Currently, robotic systems do not provide hernia tacking or advanced anastomosis stapling solutions, the training they provide their customers commonly uses suturing as the preferred approach. Further, the robotic systems also do not prevent surgeons from applying excessive loads on objects found within an instrument's jaws. It is common for surgeons to pinch synthetic hernia mesh or sutures, which induces compressive yield stress on polymers, thereby creating stress concentrations in manufactured surgical products which can become future points of product failure. Thus, there is a need for suturing improvements in surgical robotic systems.

### SUMMARY

In robotic-assisted surgery (RAS) suturing is considered a core skill that surgeons need to master and thus, spend enormous amounts of time perfecting their technique of RAS suturing. Due to challenges faced by surgeons by performing instrument exchange, it is common to see a surgeon use a single needle driver located in their dominant hand and a grasper, e.g., fenestrated bipolar grasper in their off hand. Thus, instruments that were not designed to hold and drive needles are commonly used for that purpose.

The present disclosure provides a grasper, e.g., needle driver, having electrically sensing and isolated jaws. An insulating material or housing separates a tissue contacting surface from the rest of the instrument and the jaws from each other. The insulating material may be formed from a dielectric polymer, ceramic, or composites that form an electrically non-conductive barrier between opposing jaws of the grasper. The grasper may be used with a surgical robotic system and actuated by an instrument drive unit supported on a robotic arm. The surgical robotic system may also include an impedance sensing device having an electrical power source configured to measure electrical property of the jaws, e.g., continuity, conductivity, and/or impedance. The robotic system uses the electrical property along with an angle of the jaws (i.e., open percentage) to characterize objects being gripped by the grasper. The robotic system includes a controller configured to execute software instructions embodying an algorithm for determining whether jaws of the grasper are gripping a needle and to adjust the gripping force accordingly.

According to one embodiment of the present disclosure, a surgical robotic system is disclosed. The surgical robotic system includes a robotic arm having an instrument with a pair of opposing jaws configured for grasping, and an instrument drive unit configured to couple to and to actuate the instrument. The system also includes a surgeon console having a handle controller configured to control the robotic arm and the instrument. The system further includes a controller configured to: receive an electrical property of the opposing jaws, receive an angle of the opposing jaws, determine whether the opposing jaws are grasping a metallic object, and adjust a gripping force of the opposing jaws based on the determination.

Implementations of the above embodiment may include one or more of the following features. According to one aspect of the above embodiment, the surgical robotic system may also include an electrical sensor configured to measure the electrical property. The electrical property may be impedance. In determining whether the opposing jaws are grasping the metallic object, the controller may be further configured to compare impedance to a threshold impedance corresponding to the metallic object. The metallic object may be a suturing needle. The instrument drive unit may be configured to measure the angle. In determining whether the opposing jaws are grasping the metallic object, the controller may be further configured to compare the measured angle to a threshold angle configured to grasp the needle. The controller may be further configured to determine whether the opposing jaws are grasping the suturing needle and adjust the gripping force of the opposing jaws based on the determination. The controller may be further configured to increase the gripping force in response to determining that the suturing needle is being grasped. The controller may be additionally configured to decrease the gripping force in response to determining that the suturing needle is not being grasped.

According to another embodiment of the present disclosure, a surgical robotic system is disclosed. The surgical robotic system includes a robotic arm having an instrument with a pair of opposing jaws configured for grasping and an instrument drive unit configured to couple to the instrument. The instrument drive unit includes at least one motor configured to actuate the opposing jaws between an open configuration and a closed configuration and a sensor configured to determine an angle between the opposing jaws. The system also includes a surgeon console having a handle controller configured to control the robotic arm and the instrument. The system further includes an electrical sensor in electrical communication with the pair of opposing jaws and configured to measure an electrical property of at least one of the jaws. The system also includes a controller configured to determine whether the opposing jaws are grasping a suturing needle based on the measured electrical property and the angle of the opposing jaws, and to adjust a gripping force of the opposing jaws based on the determination.

Implementations of the above embodiment may further include one or more of the following features. According to one aspect of the above embodiment, the measured electrical property may be impedance. In determining whether the opposing jaws are grasping the suturing needle, the controller may be further configured to compare the measured impedance to a threshold impedance corresponding to the suturing needle. The sensor may be a motor encoder configured to measure rotational output of the at least one motor. The instrument drive unit may include a drive controller configured to calculate the angle based on the measured rotational output. The controller may be further configured to increase the gripping force in response to determining that the suturing needle is being grasped. The controller may be further configured to decrease the gripping force in response to determining that the suturing needle is not being grasped.

According to a further embodiment of the present disclosure, a method for controlling a surgical robotic system is disclosed. The method includes measuring an electrical property of opposing jaws of an instrument actuated by an instrument drive unit, which includes a motor. The method also includes measuring an angle of the opposing jaws. The method further includes determining, at a controller, whether the opposing jaws are grasping a suturing needle based on the measured electrical property and the measured angle of the opposing jaws. The method additionally includes adjusting torque of the motor to adjust a gripping force of the opposing jaws based on the determination.

Implementations of the above embodiment may also include one or more of the following features. According to one aspect of the above embodiment, the method may also include increasing the gripping force in response to determining that the suturing needle is being grasped. The method may further include decreasing the gripping force in response to determining that the suturing needle is not being grasped. The measured electrical property may be impedance, and the method may additionally include comparing the measured impedance to a threshold impedance corresponding to the suturing needle.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a top, perspective view of an end effector, according to an embodiment of the present disclosure, for use in the surgical robotic system of FIG. 1;

FIGS. 8A-C are side views at different angle sizes of the end effector according to an embodiment of the present disclosure;

FIG. 9 is a perspective view, with parts disassembled, of the end effector of FIGS. 8A-C; and

FIG. 10 is a flow chart of a method of controlling the surgical robotic system of FIG. 1 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate a torque commands for activating one or more actuators of the robotic arm, which would, in turn, move the robotic arm in response to the movement commands.

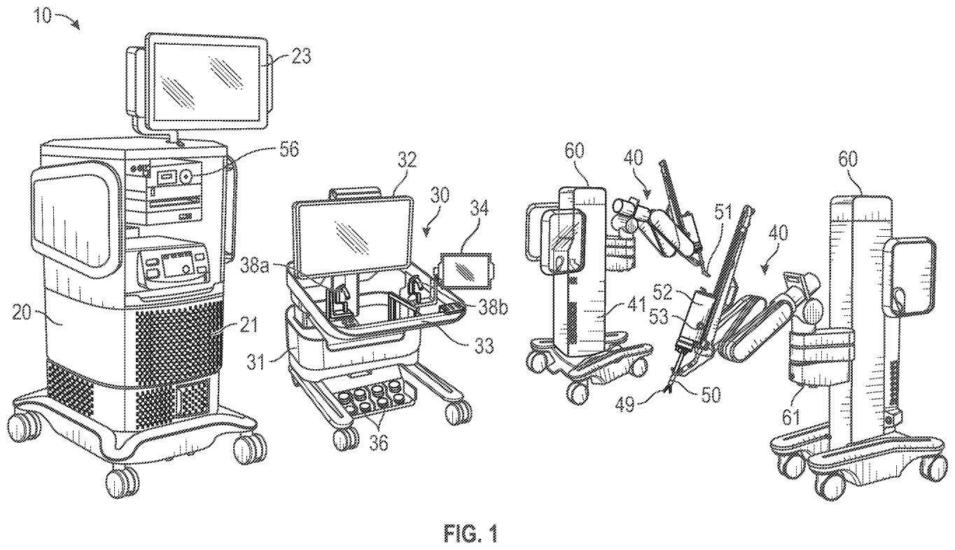

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

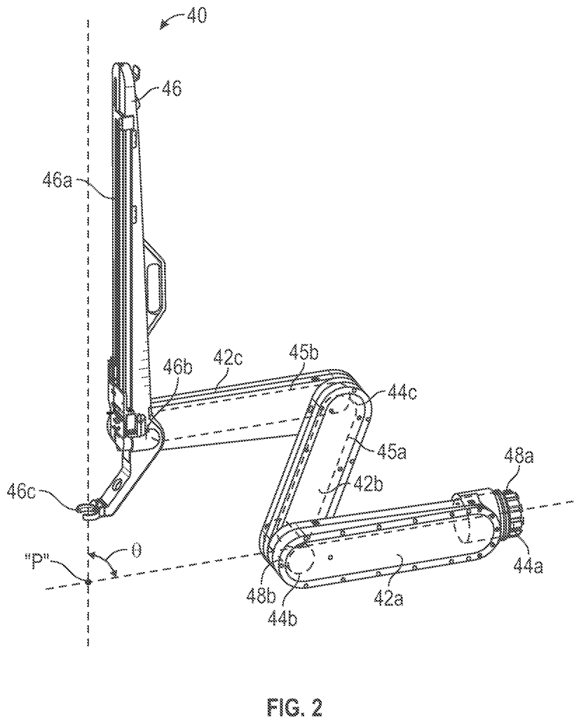

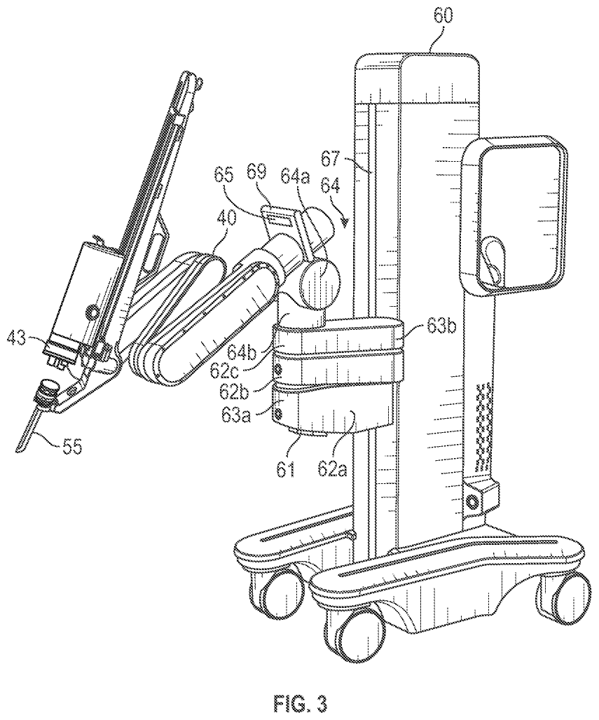

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

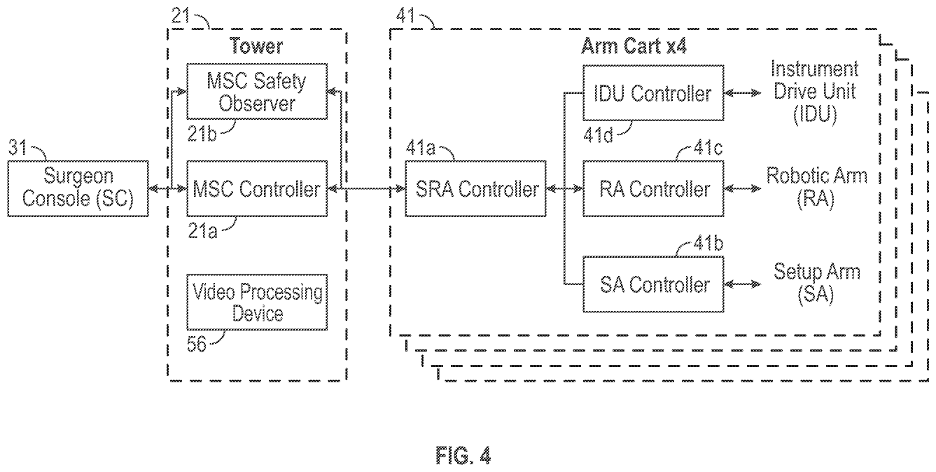

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

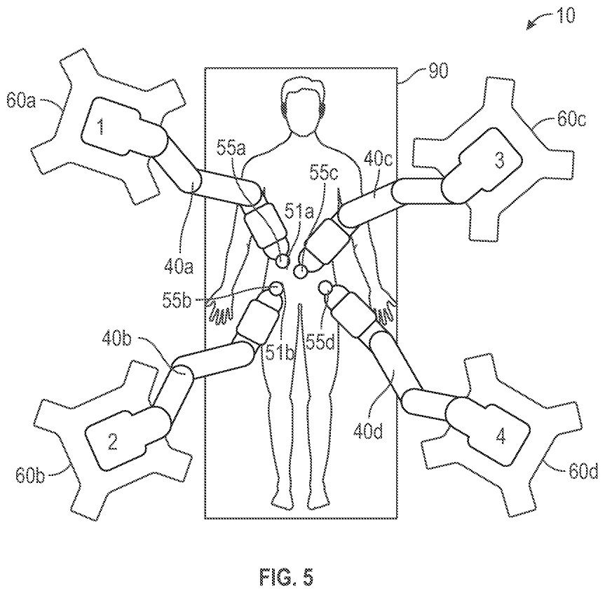

With reference to FIG. 5, the surgical robotic system 10 is setup around a surgical table 100. The system 10 includes movable carts 60a-d, which may be numbered “1” through “4.” During setup, each of the carts 60a-d are positioned around the surgical table 100. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

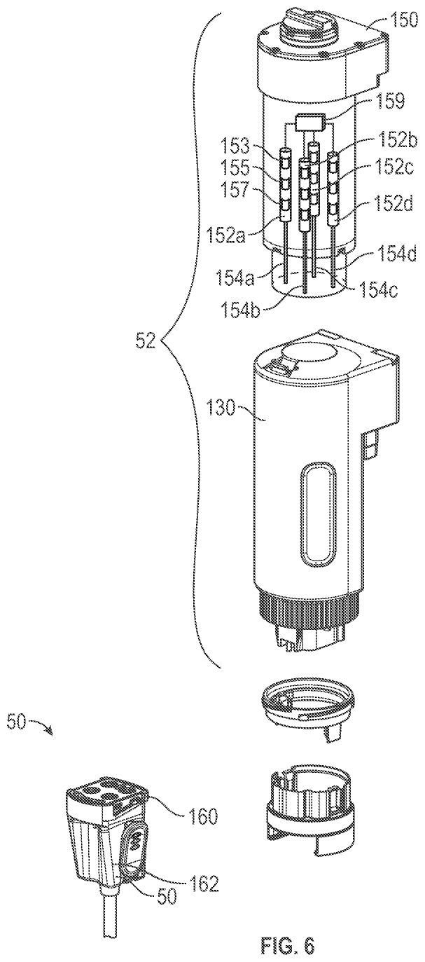

With reference to FIG. 6, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

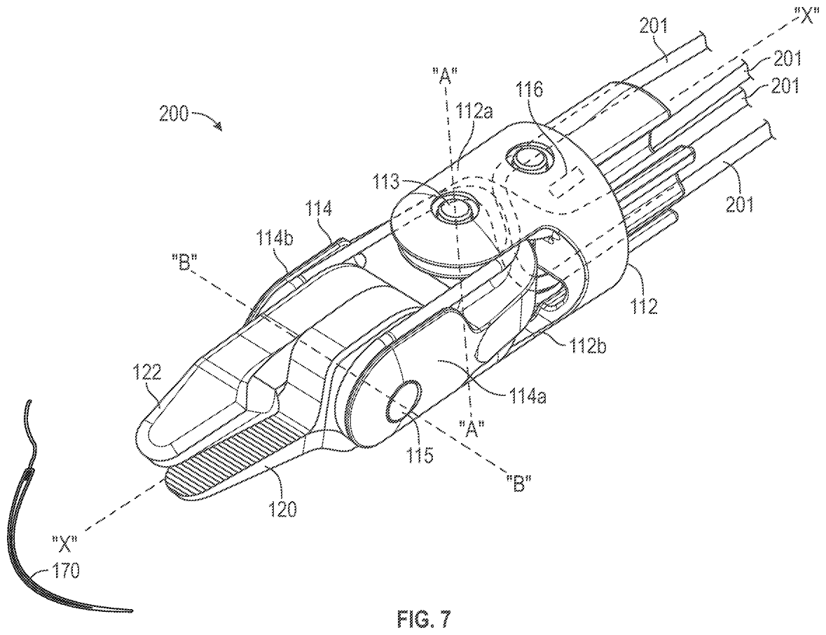

The IDU 52 includes a motor pack 150 and a sterile barrier housing 130. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 200 (FIG. 7).

Each of the motors 152a, 152b, 152c, 152d includes a current sensor 153, a torque sensor 155, and an encoder sensor 157. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The encoder 157 may be any device that provides a sensor signal indicative of the number of rotations of the motor 152a, such as a mechanical encoder or an optical encoder. Parameters which are measured and/or determined by the encoder 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIG. 6, instrument 50 includes an adapter 160 having a housing 162 at a proximal end portion thereof and an elongated shaft 164 that extends distally from housing 162. Housing 162 of instrument 50 is configured to selectively couple to IDU 52 of robotic, to enable motors 152a, 152b, 152c, 152d of IDU 52 of robotic surgical assembly 100 to operate the end effector 200 of the instrument 50. Housing 162 of instrument 50 supports a drive assembly that mechanically and/or electrically cooperates with motors 152a, 152b, 152c, 152d of IDU 52 of robotic surgical assembly 100. Drive assembly of instrument 50 may include any suitable electrical and/or mechanical component to effectuate driving force/movement.

The surgical instrument also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc.

As shown in FIG. 7, the end effector 200 may include a pair of opposing jaws 120 and 122 that are movable relative to each other. In embodiments, the end effector 200 may include a proximal portion 112 having a first pin 113 and a distal portion 114. The end effector 200 may be actuated using a plurality of cables 201 routed through proximal and distal portions 112 and 114 around their respective pulleys 112a, 112b, 114a, 114b, which are integrally formed as arms of the proximal and distal portions 112 and 114. In embodiments, the end effector 200, namely, the distal portion 114 and the jaws 120 and 122, may be articulated about the axis “A-A” to control a yaw angle of the end effector with respect to a longitudinal axis “X-X”. The distal portion 114 includes a second pin 115 with a pair of jaws 120 and 122 pivotably coupled to the second pin 115. The jaws 120 and 122 configured to pivot about an axis “B-B” defined by the second pin 115 allowing for controlling a pitch angle of the jaws 120 and 122 as well as opening and closing the jaws 120 and 122. The yaw, pitch, and jaw angles are controlled by adjusting the tension and/or length and direction (e.g., proximal or distal) of the cables 201. The end effector 200 also includes a cable displacement sensor 116 configured to measure position of the cables 201. Thus, the end effector 200 may have three degrees of freedom, yaw, pitch, and jaw angle between jaws 120 and 122.

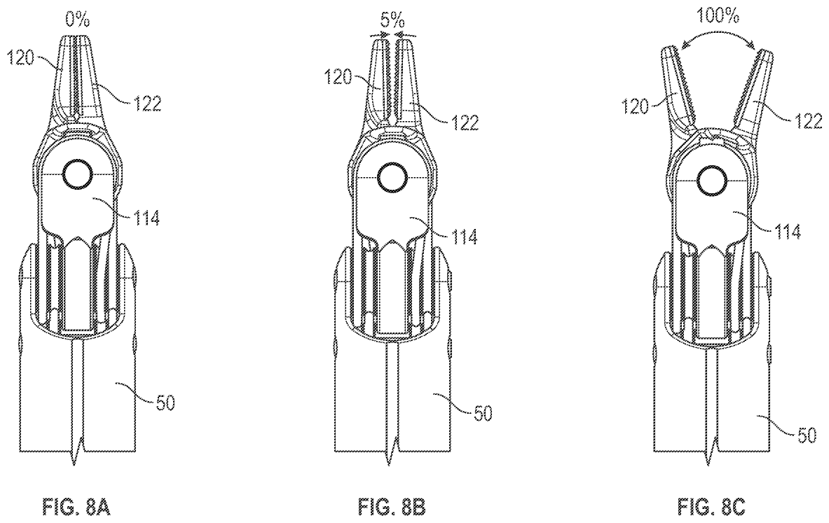

The end effector 200 may be any grasper suitable for gripping a suturing needle 170 securely and using the needle 170 during the suturing procedure (e.g., pierce tissue, pull a suture, etc.). FIGS. 8A-C show the end effector 200 in a fully closed configuration (FIG. 8A), a partially open, needle grasping configuration (FIG. 8B), and a fully open configuration (FIG. 8C). In the partially open configuration, the end effector 200 is configured to grasp the needle 170 which may have any suitable shape (e.g., straight, curved, etc.) and size (e.g., a diameter from about 0.4 mm to about 1.2 mm). The needle 170 may be formed from any metal, such as stainless steel and alloys thereof to provide for sufficient strength, ductility, and hardness during suturing procedures. Since the needle 170 is metallic, the needle 170 is electrically conductive. This property is utilized by the system 10 to determine whether the end effector 200 is grasping the needle 170 as described further below.

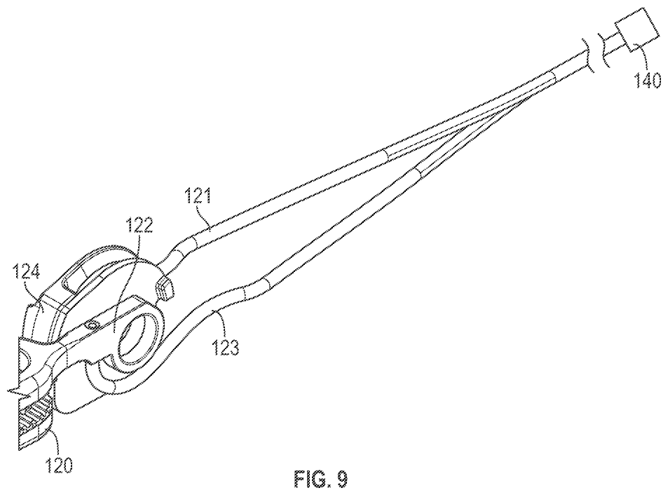

With reference to FIG. 9, each of the jaws 120 and 122 is formed from an electrically conductive material, e.g., metal, or includes a tissue-contacting surface that is electrically conductive. The jaws 120 and 122 are electrically isolated from each other by an insulator 124, which may be formed from any suitable dielectric material. The jaws 120 and 122 are coupled via a pair of electrical leads 121 and 123 to an electrical sensor 140, configured to provide an electrical signal to the jaws 120 and 122 and to measure an electrical property. The electrical sensor 140 may include an electrical signal generator configured to output an electrical signal suitable for sensing electrical properties, i.e., low current and low voltage. Sensed electrical properties may include, conductivity, continuity, impedance, resistance, phase, etc., or any other property indicative of grasping a metallic object. In embodiments, the electrical sensor 140 may be an electrosurgical generator, which includes built-in sensing capabilities.

The electrical signal may be continuous or periodic and may have a duty cycle of 100% or less. The signal may be used by the electrical sensor 140 to detect whether the jaws 120 and 122 are in an open condition and not grasping any object, which corresponds to an open circuit. Thus, while the jaws 120 and 122 are open and not contacting each other either directly or through contact with an object/tissue, the electrical circuit with the electrical sensor 140 is open. Conversely, when the jaws 120 and 122 are closed on the tissue and/or the needle 170 or any other material, the electrical sensor 140 is configured to measure the impedance thereof. In embodiments, the electrical sensor 140 may measure continuity of the electrical circuit formed by the jaws 120 and 122 and other electrical properties.

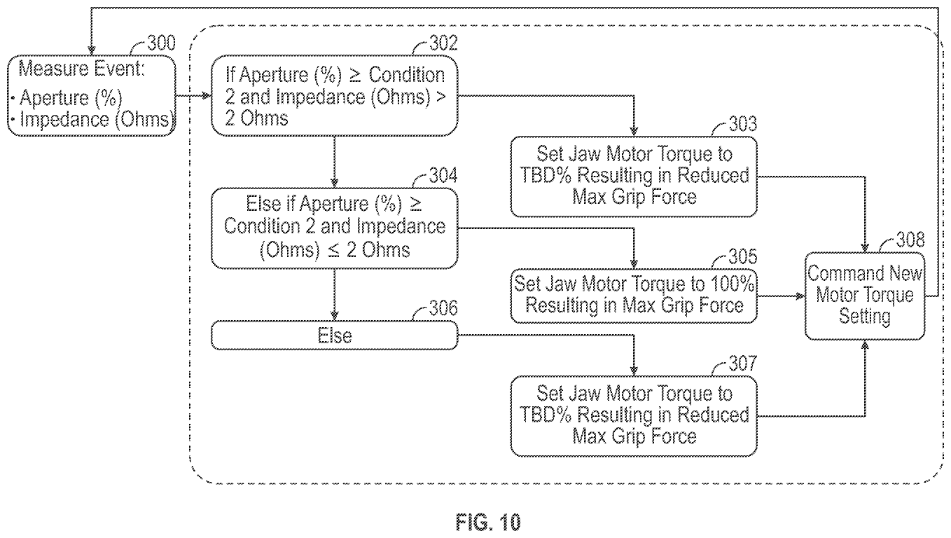

With reference to FIG. 10, a method of controlling gripping force of the end effector 200 during suturing is disclosed. The method may be implemented as software instructions executable by any controller (e.g., controller 21a, 31a, 41a, etc.). At step 300, the system 10 measures impedance and angle of the end effector 200. Measurements may be done continuously at a rate of about every 10 millisecond (ms) or above. Impedance is measured by the electrical sensor 140 as described above and angle is measured by the encoders 157, which measure rotational output of the motors 152a-d allowing the IDU controller 41d to calculate the angle of the jaws 120 and 122 by converting rotational output into linear output of the cables or drive shafts actuating the jaws 120 and 122, which corresponds to the angle.

At step 302, the controller 21a compares the measured angle to an angle threshold corresponding to the needle 170 grasping configuration. In embodiments, the angle threshold may be a range e.g., from about 5% to about 10% of the fully open configuration since an angle above a certain threshold may be indicative of grasping an object, e.g., tissue, other than the needle 170. The controller 21a also compares the measured impedance to an impedance threshold corresponding to contacting a metallic material. The impedance threshold may be from about 0 ohm to about 5 ohms, which is indicative of any electrically-conductive material. If the measured impedance is above the impedance threshold and the measured angle is at or above the angle threshold then at step 303, the controller 21a, i.e., via the IDU controller 41a, sets the torque of the motors 152a-d to less than 100% of maximum torque to reduce grip force of the end effector 200. In embodiments, the torque may be set to less than 50% of the maximum torque. The measured impedance being above the threshold indicates that the jaws 120 and 122 are not gripping a needle and are likely gripping a nonmetallic, less conductive material, e.g., tissue, suture, surgical mesh, etc. The reduction in torque that is applied to the jaws 120 and 122 allows for more delicate manipulation of tissue.

At step 304, the controller 21a determines if the measured impedance is below the impedance threshold and the measured angle is at or above the angle threshold, or alternatively, is within an angle range indicating gripping of the needle 170. If true, then at step 305 the controller 21a has identified that the jaws 120 and 122 are gripping the needle 170, since the impedance is below the threshold indicating that the conductive, i.e., metallic, object and the angle is indicative of gripping the needle 170. Thus, the controller 21a sets the torque of the motors 152a-d to 100% of maximum torque to provide for maximum gripping force of the jaws 120 and 122.

At step 306, the controller 21a determines if the measured angle is below the angle threshold, i.e., the jaws 120 and 122 are too close together to be able to grip the needle 170, then at step 307, the controller 21a, i.e., via the IDU controller 41a, sets the torque of the motors 152a-d to less than 100% of maximum torque to reduce grip force of the end effector 200, same as in step 305. In embodiments, the torque may be set to less than 30% of the maximum torque.

Following each of the steps 303, 305, 307, the motors 152a-d are operated at the torque settings set by the controller 21a at step 308. Thus, as the needle 170 is grasped, the controller 21a determines this based on the angle and electrical property of the jaws 120 and 122. The controller 21a then increases the gripping force such that end effector 200 can use the needle 170 to suture tissue. In particular, the needle 170 is gripped securely as the needle 170 is used to pierce and suture tissue to prevent dropping or slippage of the needle 170. Once the controller 21a releases the needle 170, the controller 21a decreases the gripping force, allowing for gentle manipulation of objects, until the needle 170 is gripped by the jaws 120 and 122.

This electrical sensing by the electrical sensor 140 allows for the system 10 to dynamically change the torque based on the measured angle and impedance to apply powerful grip or delicate grip depending on what the jaws 120 and 122 are grasping. Thus, if tissue or sutures are gripped, the applied torque is limited, thereby reducing the grip force. Limiting the grip force prevents or reduces the likelihood of the two primary modes of suture failure from occurring. First, the system and method of the present disclosure reduce compressive yielding of surgical products, e.g., sutures, meshes, etc., gripped by the jaws 120 and 122, which may result in stress concentrations in the suture and potential failure points. Second, low force grip makes it more likely that gripped sutures would slip out of the jaws instead of breaking as they are exposed to the ultimate tensile stress of the material. Finally, dynamic adjustment of grip force, improves the durability of the grasping instrument 50 because the instrument 50 would not need to be constantly operating at maximum grasping force throughout the procedure. Maximum grasping forces are limited to when the instrument 50 is grasping needles.

While the foregoing method was described as using two parameters to determine whether a needle is being grasped, namely, an electrical property and angle of the jaws 120 and 122, it is envisioned that other parameters of the instrument 50 and the IDU 52 may also be used to confirm and/or determine the object being grasped by the jaws 120 and 122. For example, the torque applied by the motors 152a-d may be measured and used to confirm and/or determine the object being grasped, with a high torque being applied being indicative of the hardness of the object, such as a needle.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including an instrument having a pair of opposing jaws configured for grasping and an instrument drive unit configured to couple to and to actuate the instrument;
a surgeon console including a handle controller configured to control the robotic arm and the instrument; and
a controller configured to:
receive an electrical property of the opposing jaws;
receive a measurement of an angle of the opposing jaws;
determine whether the opposing jaws are grasping a metallic object based on the electrical property and the angle of the opposing jaws to form a determination; and
adjust a gripping force of the opposing jaws based on the determination.

2. The surgical robotic system according to claim 1, further comprising an electrical sensor configured to measure the electrical property.

3. The surgical robotic system according to claim 1, wherein the electrical property is impedance.

4. The surgical robotic system according to claim 3, wherein the controller is further configured to compare the impedance to a threshold impedance corresponding to the metallic object to determine whether the opposing jaws are grasping the metallic object.

5. The surgical robotic system according to claim 1, wherein the metallic object is a suturing needle.

6. The surgical robotic system according to claim 5, wherein the controller is further configured to measure the angle of the opposing jaws based on at least one of motor position, torque, or current.

7. The surgical robotic system according to claim 6, wherein the controller is further configured to compare the measured angle to a threshold angle configured to grasp the needle to determine whether the opposing jaws are grasping the metallic object.

8. The surgical robotic system according to claim 5, wherein the controller is further configured to:
determine whether the opposing jaws are grasping the suturing needle based on the electrical property and the angle of the opposing jaws; and
adjust the gripping force of the opposing jaws based on the determination.

9. The surgical robotic system according to claim 8, wherein during adjusting the gripping force, the controller is further configured to increase the gripping force in response to determining that the suturing needle is being grasped.

10. The surgical robotic system according to claim 8, wherein during adjusting the gripping force, the controller is further configured to decrease the gripping force in response to determining that the suturing needle is not being grasped.

11. A surgical robotic system comprising:
a robotic arm including:
an instrument having a pair of opposing jaws configured for grasping; and
an instrument drive unit configured to couple to the instrument, the instrument drive unit including:
at least one motor configured to actuate the opposing jaws between an open configuration and a closed configuration; and
a sensor configured to determine an angle between the opposing jaws;
a surgeon console including a handle controller configured to control the robotic arm and the instrument;
an electrical sensor in electrical communication with the pair of opposing jaws and configured to measure an electrical property thereof the opposing jaws; and
a controller configured to:
determine whether the opposing jaws are grasping a suturing needle based on the measured electrical property and the measured angle of the opposing jaws; and
adjust a gripping force of the opposing jaws based on the determination.

12. The surgical robotic system according to claim 11, wherein the electrical property is impedance.

13. The surgical robotic system according to claim 12, wherein in determining whether the opposing jaws are grasping the suturing needle, the controller is further configured to compare the impedance to a threshold impedance corresponding to the suturing needle.

14. The surgical robotic system according to claim 11, wherein the sensor is a motor encoder configured to measure rotational output of the at least one motor, and the instrument drive unit includes a drive controller configured to calculate the angle based on the rotational output.

15. The surgical robotic system according to claim 11, wherein the controller is further configured to increase the gripping force in response to determining that the suturing needle is being grasped.

16. The surgical robotic system according to claim 11, wherein the controller is further configured to decrease the gripping force in response to determining that the suturing needle is not being grasped.

17. A method for controlling a motorized instrument, the method comprising:
measuring an electrical property of opposing jaws of the motorized instrument actuated by an instrument drive unit including at least one motor;
measuring an angle of the opposing jaws;
determining, at a controller, whether the opposing jaws are grasping a suturing needle based on the measured electrical property and the measured angle of the opposing jaws; and
adjusting torque of the at least one motor actuating the opposing jaws based on the determination.

18. The method according to claim 17, further comprising increasing a gripping force of the opposing jaws in response to determining that the suturing needle is being grasped.

19. The method according to claim 17, further comprising decreasing a gripping force of the opposing jaws in response to determining that the suturing needle is not being grasped.

20. The method according to claim 17, wherein the electrical property is impedance, and the method further comprises comparing the impedance to a threshold impedance corresponding to the suturing needle.
